(19) United States
(12) Patent Application Publication
(10) Pub. No.: US 2022/0096758 A1
(43) Pub. Date: Mar. 31, 2022

(54) MODULAR IN-LINE FILTER DEVICE

(71) Applicant: CareFusion 303, Inc., San Diego, CA (US)

(72) Inventor: Jason Andrew Wine, Placentia, CA (US)

(21) Appl. No.: 17/035,058

(22) Filed: Sep. 28, 2020

(51) Int. Cl. A61M 5/38; A61M 5/168; A61M 5/165

(52) U.S. Cl. CPC A61M 5/38; A61M 2005/1652; A61M 5/165; A61M 5/16881

(57) ABSTRACT

In-line filter devices are described herein. An in-line filter device includes an inlet portion, an outlet portion, a first filter housing, a first flow path, and a first filter media. The first filter housing defining a first filter volume. The first flow path is defined between the inlet portion and the first filter volume to provide fluid communication between the inlet portion and the first filter volume. The second flow path is defined between the first filter volume and the outlet portion to provide fluid communication between the first filter volume and the outlet portion. The first filter media is disposed within the first filter volume, wherein the first filter media permits flow from the first flow path to the second flow path and captures particulate from the flow.

### FIELD OF THE INVENTION

The present disclosure generally relates to filters, and, in particular, to filters for intravenous sets.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag. During operation, medical fluid can be filtered to prevent the transfer of bacteria, microorganisms, and/or other pathogens. Further, in certain operations, additional medical fluids or treatments can be administered to the patient via an injection port.

In some applications, multiple components or devices are utilized for the administration and filtration of medical fluids, requiring multiple tubing connections and assembly of the various components.

### SUMMARY

The disclosed subject matter relates to IV filters. In certain embodiments, an in-line filter device is disclosed that comprises an inlet portion; an outlet portion; a first filter housing defining a first filter volume; a first flow path defined between the inlet portion and the first filter volume to provide fluid communication between the inlet portion and the first filter volume; a second flow path defined between the first filter volume and the outlet portion to provide fluid communication between the first filter volume and the outlet portion; and a first filter media disposed within the first filter volume, wherein the first filter media permits flow from the first flow path to the second flow path and captures particulate from the flow.

In certain embodiments, a method is disclosed that comprises directing flow from a modular inlet body to through a first filter media disposed in a first filter volume and into an outlet portion; and capturing particulate from the flow in the first filter media disposed in the first filter volume.

In certain embodiments, an in-line filter device is disclosed that comprises an inlet portion; an outlet portion; a first filter housing defining a first filter volume; a first flow path defined between the inlet portion and the first filter volume to provide fluid communication between the inlet portion and the first filter volume; a second flow path defined between the first filter volume and the outlet portion to provide fluid communication between the first filter volume and the outlet portion; a first filter media disposed within the first filter volume, wherein the first filter media permits flow from the first flow path to the second flow path and captures particulate from the flow; a second filter housing defining a second filter volume; a third flow path defined between the inlet portion and the second filter volume to provide fluid communication between the inlet portion and the second filter volume; a fourth flow path defined between the second filter volume and the outlet portion to provide fluid communication between the second filter volume and the outlet portion; and a second filter media disposed within the second filter volume, wherein the second filter media permits flow from the third flow path to the fourth flow path and captures particulate from the flow.

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a patient receiving an infusion of a medical fluid using an IV pump.

FIG. 2 illustrates a perspective view of an in-line filter device according to certain aspects of the present disclosure.

FIG. 3 illustrates a front view of the in-line filter device of FIG. 2.

FIG. 4 illustrates a top view of the in-line filter device of FIG. 2.

FIG. 5 illustrates a bottom view of the in-line filter device of FIG. 2.

FIG. 6 illustrates a cross-sectional view of the in-line filter device of FIG. 4 along section line A-A with an inlet flow shown.

FIG. 7 illustrates a cross-sectional view of the in-line filter device of FIG. 4 along section line B-B.

FIG. 8 illustrates a cross-sectional view of the in-line filter device of FIG. 4 along section line C-C.

FIG. 9 illustrates a perspective view of the in-line filter device of FIG. 2 with the filter cover removed.

FIG. 10 illustrates a cross-sectional view of the in-line filter device of FIG. 4 along section line A-A with an outlet flow shown.

### DETAILED DESCRIPTION

The disclosed in-line filter device incorporates a modular construction. The modular construction of the in-line filter device can allow a single in-line device allow for the filtration of medical fluids and the administration of medical fluids via an injection port. By integrating the functionality of an in-line filtration device, an injection port, and a check valve, the in-line filter device can reduce complexity, assembly time, and the number of tubing connections required in the IV set.

The detailed description set forth below is intended as a description of various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding. Reference numbers may have letter suffixes appended to indicate separate instances of a common element while being referred to generically by the same number without a suffix letter.

While the following description is directed to in-line components for the administration of medical fluid using the disclosed in-line device, it is to be understood that this description is only an example of usage and does not limit the scope of the claims. Various aspects of the disclosed in-line device may be used in any application where it is desirable to provide modular device construction to reduce complexity and tubing connections.

The disclosed in-line filter device overcomes several challenges discovered with respect to certain conventional in-line devices. One challenge with certain conventional in-line devices is that separate components are often used for filtration, injection sites, and check valve functionality, adding complexity to IV sets and adding to the number of tubing connections that are required in an IV set. For example, an IV set that includes a separate filter, injection site, and check valve assembly can require up to 6 tubing connections, increasing assembly time and the potential for leaks or faulty connections. Further, another challenge with certain conventional in-line filter devices is that filter devices often require inverting the filter device during priming to purge air from the device. Additionally, certain conventional in-line devices may be difficult for a clinician to handle. Because certain conventional in-line devices may add complexity to an IV set, may be difficult to prime, and may be difficult for a clinician to handle, the use of certain conventional in-line devices is undesirable.

Therefore, in accordance with the present disclosure, it is advantageous to provide an in-line devices as described herein that allows for integration of the injection site, check-valve, and filter functionality into a single in-line component, reducing complexity and tubing connections. Further, it is advantageous to provide an in-line device that allows for easier priming operations and that is easy to handle.

Examples of in-line devices that minimize tubing connections and allow for various configurations are now described.

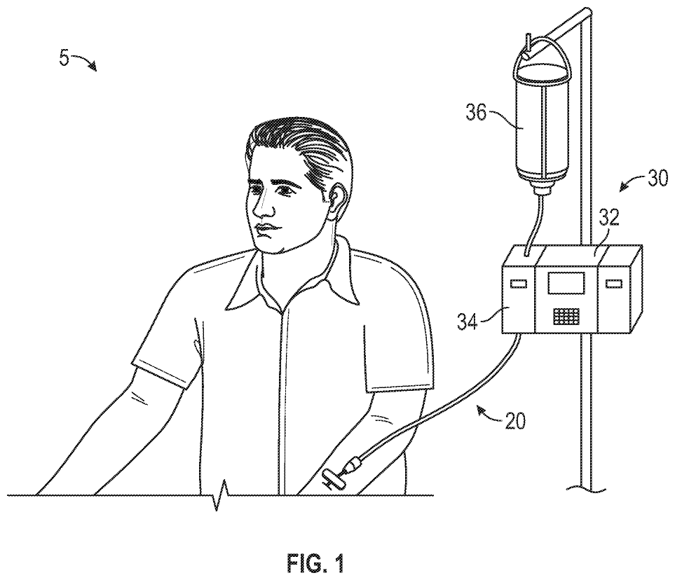

FIG. 1 illustrates a patient 5 receiving an infusion of a medical fluid through an IV pump 30 according to certain aspects of the present disclosure. The IV pump 30 comprises a controller 32 and two pump modules 34. An IV set 20 is connected between a container 36 of the medical fluid and the patient 5. During operation, medical fluid delivered to the patient 5 can flow through an in-line device that can allow medical fluid to be filtered, additional fluid to be introduced via an injection port, and/or prevent the back flow of fluid into the container 36. In some embodiments, the in-line device can be disposed in between or in line with tubing of the IV set 20.

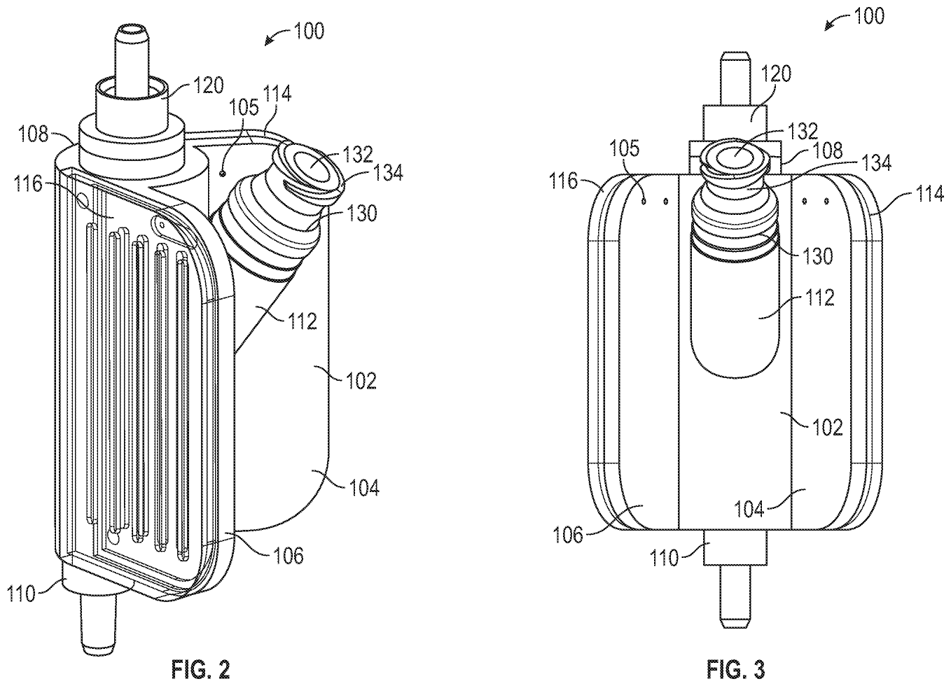

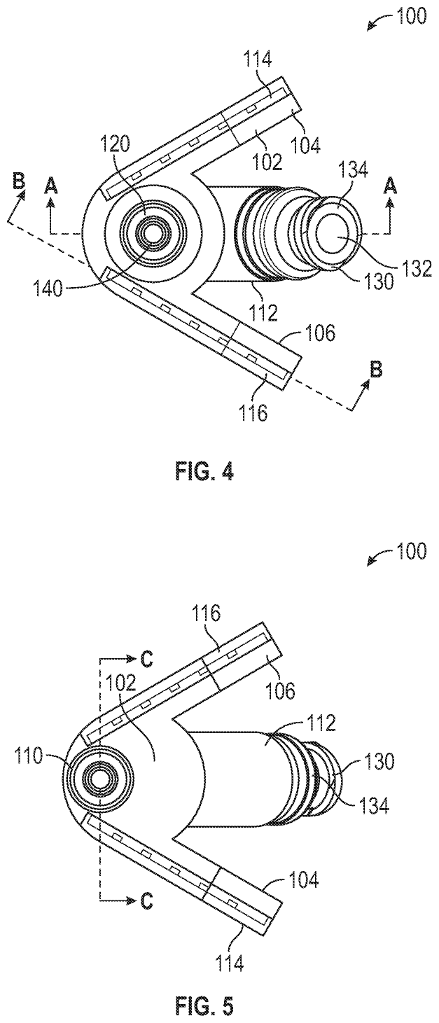

FIG. 2 illustrates a perspective view of an in-line filter device 100 according to certain aspects of the present disclosure. FIG. 3 illustrates a front view of the in-line filter device 100 of FIG. 2. FIG. 4 illustrates a top view of the in-line filter device 100 of FIG. 2. FIG. 5 illustrates a bottom view of the in-line filter device 100 of FIG. 2. In the depicted example, the in-line filter device 100 allows flow from the IV set 20 to pass therethrough.

As illustrated, fluid flow enters the in-line filter device 100 through an inlet 120 extending from a housing 102 of the in-line filter device 100. The inlet 120 can extend away from the housing 102. The inlet 120 can provide fluid communication with flow paths defined within the housing 102, permitting fluid flow to enter the in-line filter device 100. The housing 102 can be formed from a rigid material, including, but not limited to plastic.

In some embodiments, the inlet 120 can be a modular component that is releasably attached to the housing 102. As illustrated, the inlet 120 can be attached to an inlet base 108 formed in the housing 102. The inlet base 108 can be formed to provide a sealing engagement with the inlet 120. The inlet base 108 can provide fluid communication with the flow paths defined within the in-line filter device 100. Advantageously, the inlet base 108 can receive various inlets to configure the functionality of the in-line filter device 100. For example, the inlet base 108 can receive different inlets 120 that are configured to engage with or interface with various connectors or tubing of the IV set 20.

Optionally, as described herein, the inlet 120 can include a check valve 140 to allow inlet flow through the inlet 120 while preventing back flow from the in-line filter device 100 from flowing back through the inlet 120 and through the IV set 20. In some embodiments, the check valve 140 can be a movable member that moves to a flow position to permit flow into the in-line filter device 100 and to a seal position to prevent back flow out of the in-line filter device 100 via the inlet 120. The check valve 140 can be a biased member.

During operation, fluid introduced into the in-line filter device 100 can pass through one or more filters disposed in filter housings 104, 106. Optionally, the filter housings 104, 106 can include filter covers 114, 116 to enclose the filters within the filter housings 104, 106. The filter covers 114, 116 may be removable to provide access to the filters. Further, the filter housings 104, 106 can allow a clinician to easily handle or position the in-line filter device 100.

The filters can prevent the transfer of bacteria, microorganisms, and/or other pathogens to the patient. As described herein, fluid can flow from the inlet 120 through the filters and to the outlet 110. During operation, a positive pressure differential can direct fluid flow from the inlet 120 through the filters to the outlet 110.

In the depicted example, fluid flow can flow out of the in-line filter device 100 into the IV set 20 through an outlet 110 extending from the housing 102. The outlet 110 can extend away from the housing 102. The outlet 110 can provide fluid communication with flow paths defined within the housing 102, permitting fluid flow to exit the in-line filter device 100.

Optionally, the in-line filter device 100 can allow for a supplemental fluid or treatment to be injected into the in-line filter device 100 without interrupting the flow through the IV set 20. In some embodiments, a supplemental fluid can be injected by a syringe into the in-line filter device 100 through an injection port 130 extending from a housing 102 of the in-line filter device 100. The injection port 130 can extend away from the housing 102. The injection port 130 can provide fluid communication with flow paths defined within the housing 102, permitting fluid flow to enter the in-line filter device 100. Similar to fluid flow through the inlet 120, fluid injected into the in-line filter device 100 can be filtered by the in-line filter device 100.

In some embodiments, the injection port 130 can be a modular component that is releasably attached to the housing 102. As illustrated, the injection port 130 can be attached to an injection port base 112 formed in the housing 102. injection port base 112 can be formed to provide a sealing engagement with the injection port 130. The injection port base 112 can provide fluid communication with the flow paths defined within the in-line filter device 100. Advantageously, the injection port base 112 can receive various injection ports to configure the functionality of the in-line filter device 100. For example, the injection port base 112 can receive different injection ports 130 that are configured to engage with or interface with various syringes or other fluid delivery devices.

As can be appreciated, the in-line filter device 100 can allow for the functionality of an in-line filtration device, a check valve, and/or an injection port in a single in-line device. Advantageously, by allowing multiple functions to be integrated into the in-line filter device 100, the complexity and number of tubing connections between components can be reduced. Further, the in-line filter device 100 can be configured to provide application specific functionality, reducing complexity.

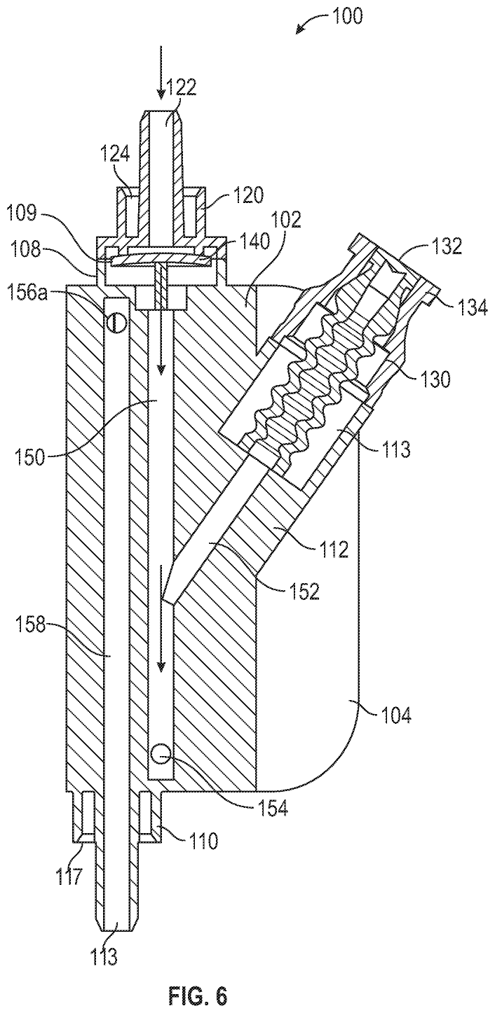

FIG. 6 illustrates a cross-sectional view of the in-line filter device 100 of FIG. 4 along section line A-A with an inlet flow shown. As illustrated, the housing 102 of the in-line filter device 100 defines a plurality of flow paths therein to direct the flow of fluid through the in-line filter device 100.

As described herein, fluid flow enters the in in-line filter device 100 through the inlet 120. As illustrated, the body of the inlet 120 can define an inlet lumen 122 to provide fluid communication with an inlet flow path 150 defined within the housing 102, permitting fluid flow to enter the in-line filter device 100.

In some embodiments, tubing from the IV set 20 can be coupled to the inlet 120 to allow flow from a fluid container 36 or other component of the IV set 20 into the inlet flow path 150 defined within the housing 102. Optionally, the tubing or a connector attached to the tubing can engage with or be disposed within a recess or groove 124 formed around the inlet 120.

Optionally, the check valve 140 can prevent back flow from the inlet flow path 150 from flowing back through the inlet lumen 122. As illustrated, the check valve 140 can be disposed between the inlet 120 and the inlet flow path 150 to control flow between the inlet lumen 122 and the inlet flow path 150. In some embodiments, the check valve 140 is disposed in a recess 109 formed in the inlet base 108. During operation, the check valve 140 can move or deform to a flow position to permit inlet flow from the inlet lumen 122 to the inlet flow path 150. Further, the check valve 140 can move or deform to a sealing position, preventing back flow from the inlet flow path 150 back through the inlet lumen 122. The check valve 140 can be a deformable disk that moves or deforms in response to flow direction/pressures.

As illustrated, the inlet flow can flow through the housing 102 via the inlet flow path 150. The inlet flow path 150 can direct the inlet flow from the inlet 120 to the filters disposed within the filter housings 104, 106 via filter port 154. In some embodiments, the filter port 154 can direct inlet flow to both filter housings 104, 106. Optionally, the filter port 154 can direct the inlet flow to a single filter disposed in a single filter housing 104 of the in-line filter device 100.

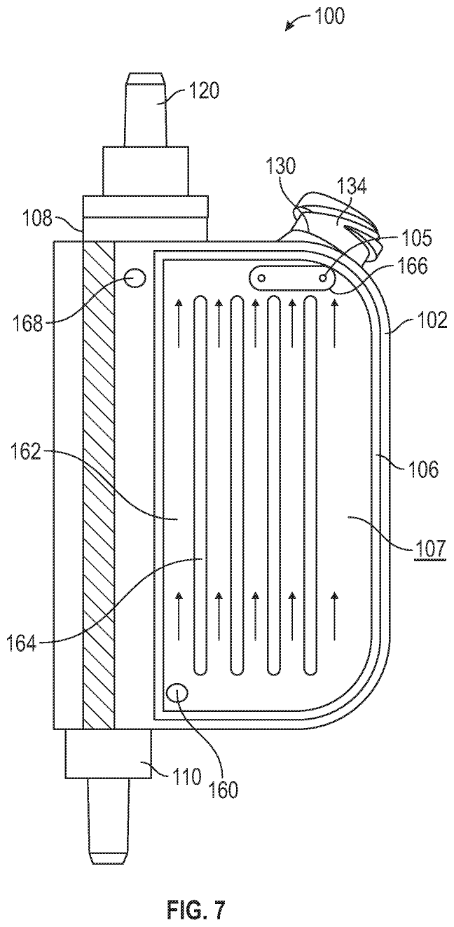

FIG. 7 illustrates a cross-sectional view of the in-line filter device 100 of FIG. 4 along section line B-B. As illustrated, flow from the filter port 154 flows into the filter housing 106 via the filter inlet port 160. The fluid flow from the filter inlet port 160 enters the volume 107 defined by the filter housing 106 (and the optional filter cover). As can be appreciated, flow from the filter port 154 can enter the filter housing 104 via a similar filter inlet port 160. In some embodiments, the filter housing 104 can have a similar structure or configuration as described with respect to the filter housing 106.

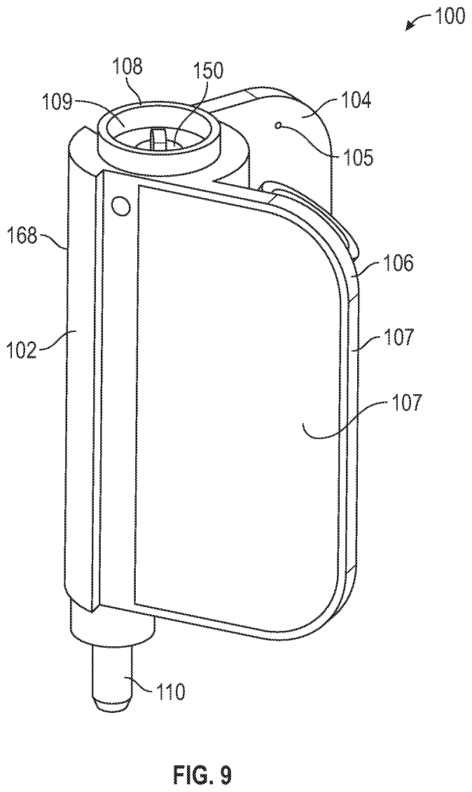

As illustrated, fluid within the volume 107 can pass through a filter media (shown in FIG. 9) to prevent the transfer of bacteria, microorganisms, and/or other pathogens to the patient. During operation, fluid can flow from the inlet port 160 in an inlet portion of the volume 107 through the filter media to the outlet portion of the volume 107. As can be appreciated, a positive pressure differential can direct fluid flow from the inlet portion of the volume 107 through the filter media to the outlet portion of the volume 107. During operation, fluid can flow from the outlet portion of the volume 107 to a filter outlet port 168 in fluid communication with the outlet portion of the volume 107.

In some embodiments, the filter media can be supported by portions of the filter housing 106. As illustrated, the filter housing 106 can define one or more filter supports 164 extending along the length of the filter housing 106 to space the filter media apart from the filter housing 106. The filter supports 164 can be spaced apart to define flow channels 162 between the filter media and the filter housing 106, allowing fluid flow in the volume 107 to enter the filter media along the surface area of the filter media. Optionally, the filter media is captured between the filter cover and the filter supports 164.

In some embodiments, the filter housing 106 can include one or more vent holes 105 to allow gases to be purged from the volume 107. During operation, as fluid enters the in-line filter device 100, the vent holes 105 can allow trapped gasses to escape via the vent holes 105. Optionally, the vent holes 105 can allow gasses trapped by a hydrophilic filter media to be vented or purged. Advantageously, the vent holes 105 can allow the in-line filter device 100 to be primed without inverting the device to remove any trapped gasses.

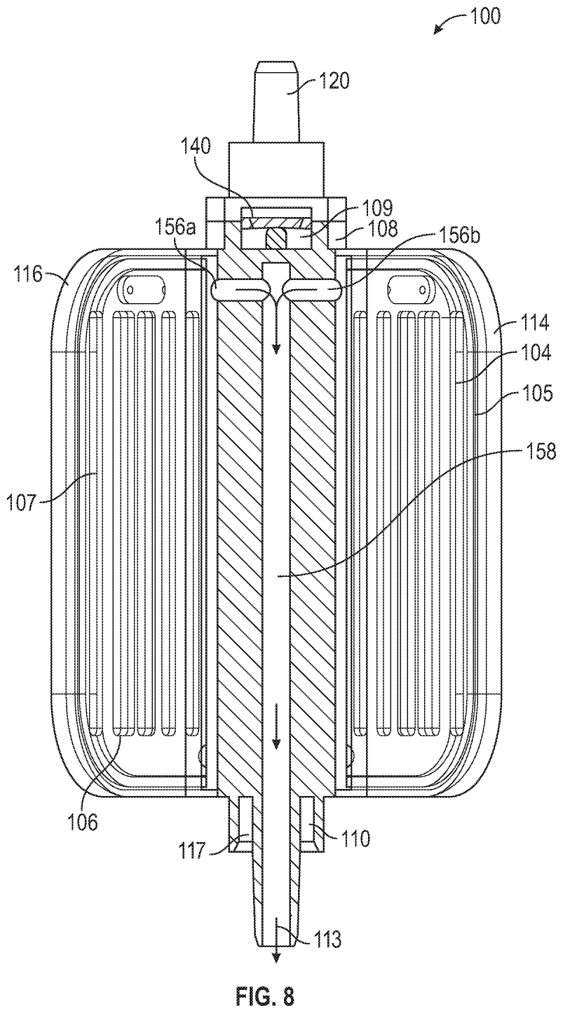

FIG. 8 illustrates a cross-sectional view of the in-line filter device 100 of FIG. 4 along section line C-C. As illustrated, filtered flow from the filter housing 106 can exit the in-line filter device 100 through the outlet 110 of the in-line filter device 100. During operation, filtered flow from the filter housing 106 can flow from a filter port 156a through an outlet flow path 158 to the outlet 110. Similarly, filtered flow from the filter housing 104 can flow from a filter port 156b through the outlet flow path 158 to the outlet 110. In some embodiments, the body of the outlet 110 can define an outlet lumen 113 to provide fluid communication with the outlet flow path 158, permitting filtered flow to exit the in-line filter device 100.

In some embodiments, tubing from the IV set 20 can be coupled to the outlet 110 to allow flow from the in-line filter device 100 to flow into a patient directly or into other components of the IV set 20. Optionally, the tubing or a connector attached to the tubing can engage with or be disposed within a recess or groove 117 formed around the outlet 110.

FIG. 9 illustrates a perspective view of the in-line filter device 100 of FIG. 2 with the filter cover 116 removed. As described herein, fluid within the volume 107 can pass through a filter media 170 to prevent the transfer of bacteria, microorganisms, and/or other pathogens to the patient. During operation, the filter media 170 can selectively filter the flow through the in-line filter device 100. The filter media 170 can have an average filter opening of approximately 0.2 microns. Optionally, the average filter opening of the filter media 170 can range between 0.1 microns and 10 microns. In some embodiments, the filter media 170 can be formed from a non-woven filter material. The filter media 170 can be formed from a resilient or expandable material. The filter media 170 can be a hydrophilic membrane.

The filter media 170 can have a generally planar or rectangular prism shape. As illustrated, the filter media 170 can extend along a portion of the width and length of the filter housing 106. In some embodiments, the filter media 170 can extend generally along the width of the filter housing 106. During operation, fluid flow can flow into the filter media 170 along the surface area of the filter media 170 exposed to the inlet flow.

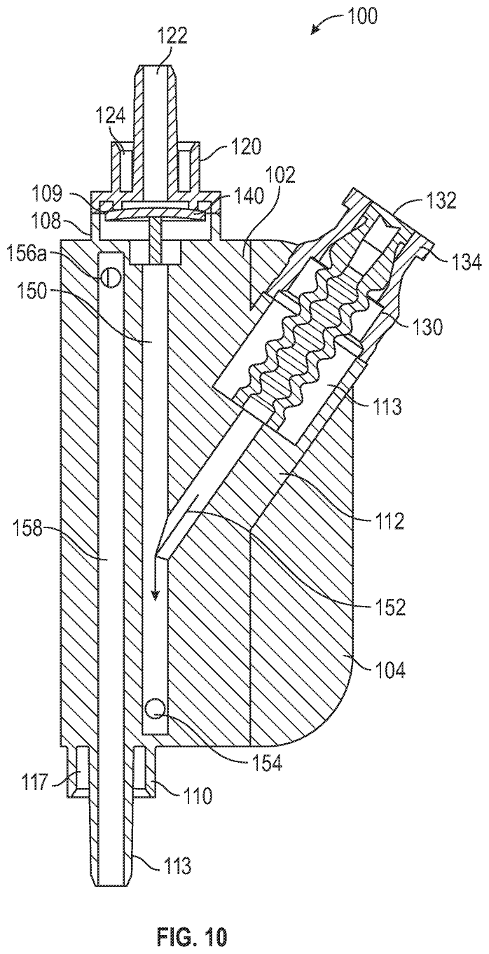

FIG. 10 illustrates a cross-sectional view of the in-line filter device 100 of FIG. 4 along section line A-A with an outlet flow shown. As described herein, the in-line filter device 100 can allow for a supplemental fluid or treatment to be injected into the in-line filter device 100 through the injection port 130 of the in-line filter device 100.

As illustrated, the port body 134 can define a sealable port 132 that is in fluid communication with an injection flow path 152 defined within the housing, permitting supplemental fluid flow to enter the in-line filter device 100. Various syringes or connectors, including needless connectors can be coupled or engaged with the injection port 130 to allow flow from a delivery device into the injection flow path 152 defined within the housing 102. In some embodiments, fluid injected into the injection flow path 152 can join the inlet flow path 150 to be filtered by the in-line filter device 100.

Optionally, the check valve 140 can prevent injection flow from flowing through the inlet 120. During operation, the check valve 140 can prevent back flow from the injection flow path 152, directing the supplemental flow into the filters via the filter port 154. Further, the check valve 140 can prevent air from entering the IV set during an injection.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An in-line filter device, comprising:
an inlet portion;
an outlet portion;
a first filter housing defining a first filter volume;
a first flow path defined between the inlet portion and the first filter volume to provide fluid communication between the inlet portion and the first filter volume;
a second flow path defined between the first filter volume and the outlet portion to provide fluid communication between the first filter volume and the outlet portion; and
a first filter media disposed within the first filter volume, wherein the first filter media permits flow from the first flow path to the second flow path and captures particulate from the flow.

2. The in-line filter device of claim 1, wherein the inlet portion defines an inlet base.

3. The in-line filter device of claim 2, wherein the inlet base is configured to receive a modular inlet body.

4. The in-line filter device of claim 2, wherein the inlet base is configured to receive a modular check valve member.

5. The in-line filter device of claim 1, further comprising:
an injection port portion; and
a third flow path defined between the injection port portion and the first filter volume to provide fluid communication between the injection port portion and the first filter volume.

6. The in-line filter device of claim 5, wherein the injection port portion defines an injection port base.

7. The in-line filter device of claim 6, wherein the injection port base is configured to receive a modular injection port body.

8. The in-line filter device of claim 1, wherein the first filter media comprises a hydrophilic material.

9. The in-line filter device of claim 1, wherein the first filter housing comprises a plurality of filter supports configured to engage the first filter media.

10. The in-line filter device of claim 1, wherein the first filter housing defines a vent port in fluid communication with the first filter volume.

11. The in-line filter device of claim 1, wherein the first filter housing comprises a first filter cover, the first filter cover cooperatively defining the first filter volume.

12. The in-line filter device of claim 1, further comprising:
a second filter housing defining a second filter volume;
a fourth flow path defined between the inlet portion and the second filter volume to provide fluid communication between the inlet portion and the second filter volume;
a fifth flow path defined between the second filter volume and the outlet portion to provide fluid communication between the second filter volume and the outlet portion; and
a second filter media disposed within the second filter volume, wherein the second filter media permits flow from the fourth flow path to the fifth flow path and captures particulate from the flow.

13. A method comprising:
directing flow from a modular inlet body to through a first filter media disposed in a first filter volume and into an outlet portion; and
capturing particulate from the flow in the first filter media disposed in the first filter volume.

14. The method of claim 13, further comprising:
injecting a fluid into the first filter volume the first filter media via a modular injection port body.

15. The method of claim 14, further comprising:
preventing backflow of the fluid from the modular injection port body into the modular inlet body via a modular check valve member disposed between the modular inlet body and the modular injection port body.

16. The method of claim 13, further comprising:
venting gas from the first filter volume via a vent port in fluid communication with the first filter volume.

17. The method of claim 13, further comprising:
directing flow from the modular inlet body to through a second filter media disposed in a second filter volume and into the outlet portion; and
capturing particulate from the flow in the second filter media disposed in the second filter volume.

18. An in-line filter device, comprising:
an inlet portion;
an outlet portion;
a first filter housing defining a first filter volume;
a first flow path defined between the inlet portion and the first filter volume to provide fluid communication between the inlet portion and the first filter volume;
a second flow path defined between the first filter volume and the outlet portion to provide fluid communication between the first filter volume and the outlet portion;
a first filter media disposed within the first filter volume, wherein the first filter media permits flow from the first flow path to the second flow path and captures particulate from the flow;
a second filter housing defining a second filter volume;
a third flow path defined between the inlet portion and the second filter volume to provide fluid communication between the inlet portion and the second filter volume;
a fourth flow path defined between the second filter volume and the outlet portion to provide fluid communication between the second filter volume and the outlet portion; and
a second filter media disposed within the second filter volume, wherein the second filter media permits flow from the third flow path to the fourth flow path and captures particulate from the flow.

19. The in-line filter device of claim 18, further comprising:
an injection port portion; and
a fifth flow path defined between the injection port portion and the first filter volume and the second filter volume to provide fluid communication between the injection port portion and the first filter volume and the second filter volume.

20. The in-line filter device of claim 19, further comprising:
a modular check valve member disposed between the injection port portion and the inlet portion, wherein the modular check valve member prevents backflow from the injection port portion to the inlet portion.
